(19) United States
(12) Patent Application Publication
(10) Pub. No.: US 2012/0148496 A1
(43) Pub. Date: Jun. 14, 2012

(54) Method of Altering Reaction Time

(76) Inventor: Alan R. Hirsch, Riverwoods, IL (US)

(21) Appl. No.: 13/314,628

(22) Filed: Dec. 8, 2011

Related U.S. Application Data
(60) Provisional application No. 61/422,110, filed on Dec. 10, 2010.

(51) Int. Cl. A61K 49/00; B65D 69/00; A61B 5/00; A61K 36/63; A61P 25/00

(52) U.S. Cl. 424/9.2; 424/725; 600/300; 206/569

(57) ABSTRACT

Methods and compositions for modifying an individual's reaction time to an external stimulus are disclosed.

### CROSS REFERENCE TO RELATED APPLICATION

This application claims the benefit of U.S. Provisional Application No. 61/422,110, filed Dec. 10, 2010, the teachings and disclosures of which are incorporated herein by reference.

### FIELD OF THE INVENTION

Embodiments of the invention relate to altering an individual's reaction time to an external stimulus by the administration of an odorant composition for inhalation by the individual.

### BACKGROUND OF THE INVENTION

Sensory modalities are the receptive windows to the environment and are known to present an integrated comprehensive map of the external milieu. Multi-sensory interactions are important for elucidation of an apt response and hence, for survival. Interactive processing of stimuli alters the efferent pathways of the brain, hence, modulating responses to environmental triggers.

Olfaction has been shown to have an effect on a wide spectrum of human affect and cognition, from time perception to the process of reproduction. Certain odors have been found to alter an individual's performance of complex tasks, arousal, mood, attention/concentration, memory retrieval, stress and interactions relating to sex and reproduction. The effect of odor on objective parameters, such as amplitude of contingent negative variation (CNV) and EEG recordings, provides evidence of the influence of olfactory perception on brain function. In addition, the effect of olfactory sensation has been shown in newborns and young children, and in the absence of conscious detection of an aroma, thus demonstrating an independence from any pre-existing bias of the test subject.

The causality of the effects of odors can be attributed to the wide array of interconnecting pathways between sensory, limbic and motor cortices. One mechanism is the activation of the cortex by the limbic system. The mesocorticolimbic system is responsible for hedonic characterization of sensory stimuli. The projections as a result of environmental stimuli stimulate the cortex, independent of the limbic system. However, the limbic connections add emotional importance to the stimuli and synergistically activate the cortex through direct stimulation of the reticular activating system. Thus, the primary stimulus acquires greater importance than the incidental events in the environment, extracting greater attention from the brain than would occur in the absence of a limbic influence. This enhanced attention level can be objectively measured as reduced reaction time in response to a stimulus.

The ability for faster reaction time is important in daily life and it would be useful to provide a means of altering an individual's reaction time that is non-invasive, convenient, safe, and easy to administer.

### DESCRIPTION OF THE INVENTION

The present invention relates to methods of using odorants to alter an individual's reaction time by the administration of a composition comprising an odorant or mixture of odorants for inhalation by the individual into his/her nasal passageway. In particular, the method involves delivering a composition composed of an effective amount or concentration of an odorant or mixture of odorants to an individual for inhaling to modify their reaction time to an external stimulus compared to their reaction time without inhaling the odorant or odorant mixture. The method can be utilized with both male and female individuals.

The method can be applied to enhance or speed up reaction time of an individual to an external stimulus, for example, to facilitate physical therapy and/or rehabilitation and enhance, for example, athletic performance. Clinical applications can include administering the composition, for example, to patients undergoing physical therapy and/or rehabilitation to improve a slow or impaired reaction time to a stimulus and to promote mental processing. In some embodiments, the methods of the invention can be used as part of therapeutic intervention in rehabilitation of brain trauma or stroke to improve (decrease) reaction times of an affected individual.

“Reaction time” is the elapsed time between onset of a sensory stimulus (e.g., a light, a vocal command, a signal, etc.) and the subsequent behavioral response by the individual. Posner, M. I., Orienting of Attention. Q. J. Exp. Psychol. 32:3-25 (1980). Reaction time, which involves the triad of perception, central integration and motor response, is used to measure the influence of olfactory stimuli on the attention process of an individual as an indicator of the individual's speed of mental processing.

In embodiments of the invention, a composition that is hedonically pleasant (positive) to an individual and composed of an effective amount of an odorant or mixture of odorants can be administered for inhalation to the individual prior to an event involving exposure to a sensory stimulus (to which they respond) to cause their reaction time to respond to the sensory stimulus to be decreased (reduced) compared to their reaction time without having inhaled the composition such that the individual more quickly responds to the sensory stimulus. Such a response can be in the form of a physical act, for example, the release/depression of a “reaction” button, a physical act or movement, a vocal response, or other action or behavior that can be observed.

In embodiments, the composition can comprise one or more odorants that the subject individual considers to be hedonically pleasant (positive). In other embodiments, the composition can comprise one or more odorants that the subject individual considers to be hedonically pleasant combined with one or more odorants that the subject individual considers to be hedonically neutral.

In embodiments of the invention, a composition that is considered to be hedonically pleasant to an individual and which comprises a hedonically pleasant odorant as the primary or dominant odor (aroma) of the composition, solely or in a mixture of odorants, can be administered to the individual to inhale, to decrease the individual's reaction time to a sensory stimulus compared to the individual's reaction time to the sensory stimulus without inhaling the hedonically pleasant composition, by a statistically significant amount (p<0.05). In some embodiments, such a mixture of odorants is composed of the primary or dominant odorant in combination with one or more secondary odorants considered to be hedonically pleasant or neutral to the subject individual.

In some embodiments, the composition comprises an effective amount of a jasmine odorant as the primary or dominant odor (aroma), alone or in combination with one or more of hedonically pleasant or neutral secondary odorants. In some embodiments, the composition consists essentially of an effective amount of a jasmine odorant.

The composition is inhaled by the subject individual prior to receiving or being exposed to a sensory stimulus to result in a reduced reaction time to the sensory stimulus compared to the individual's reaction time to the sensory stimulus without having inhaled the composition. In some embodiments, the composition is inhaled by the individual prior to and during exposure to the sensory stimulus.

As used herein, the term “odorant” refers to an odor-causing chemical compound or mixture of compounds that, when delivered in a gaseous or aerosol medium, can stimulate olfactory and/or trigeminal chemoreceptors in the nasal cavity and cause a physiological or psychological response. A hedonically pleasant or positive composition, odorant or odorant mixture is one to which the subject individual has a pleasant or positive reaction to its scent. A hedonically negative composition, odorant or odorant mixture is one to which the individual has a repulsive or negative reaction to its scent. A hedonically neutral composition, odorant or odorant mixture is one to which the individual has neither a positive nor negative reaction.

In a preferred embodiment, the subject individual is presented with the composition containing a suprathreshold concentration (e.g., about 25-55 decismel units) of the odorant or odorant mixture that is near but not so high as to become an irritant (trigeminal), which the individual inhales prior to exposure to a sensory stimulus. The level or concentration of the odorant or odorant mixture within the composition and/or mode of administering the composition is sufficient to overcome competing or conflicting ambient odors that may act to nullify its effect.

An odorant is presented at a “suprathreshold” level when the decismel level or concentration of the odorant is beyond that needed to be detected by a normosmic individual. At its irritative level, the odorant quantity is so high and intense that the odorant stimulates predominantly the trigeminal nerve (for pain) rather than the olfactory nerve and, hence, is perceived as noxious or painful. The irritation threshold of the patient is the lowest concentration of the substance that causes immediate stinging or burning sensations in the nose, or stinging or lacrimation of the eye. (See, J. F. Gent, in Clinical Measurement of Taste and Smell, pages 107-166, H. L. Meiselman et al. (eds.), 602 pp., MacMillan, N.Y. (1986); R. L. Doty et al., Ann. Neurol. 25: 166-171 (1989); E. Koss et al., Neurology 38: 1228-1232 (1988); and R. Doty, The Smell Identification Test: Administration Manual 1983: 13-14, Philadelphia: Sensonics, Inc. (1983)).

If desired, prior to the administration of the odorant, the subject individual can undergo olfactory testing according to a test such as the University of Pennsylvania Smell Identification Test (UPSIT), a 40-question forced-choice, scratch-and-sniff identification test (R. Doty, The Smell Identification Test: Administration Manual 1983: 13-14, Philadelphia: Sensonics, Inc. (1983)), the Chicago Smell Test, a 3-item detection and identification test (A. R. Hirsch et al., Chemical Senses 18(5): 570-571 (1993); A. R. Hirsch et al., Chemical Senses 17(5): 643 (1992)), the Quick Smell Identification Test (Q-SIT), a three (3) question scratch-and-sniff smell identification test (Sensonics, Inc., Haddon Heights, N.J.), or other detection and/or identification test.

The subject individual can also be evaluated for olfactory capacity (e.g. loss of smell) according to an olfactory threshold test as known and used in the art. Such a test provides a precise magnitude of loss of smell and classifies the individual as normosmic, hyposmic or anosmic, which is useful in assessing the effectiveness of a particular odorant and/or the required concentration of the odorant, preferably a suprathreshold and near but below irritant level, to provide the desired effect according to the method of the invention. In the embodiment of that test, an odorant substance such as butyl alcohol, phenyl ethyl alcohol, or pyridine, is combined in an odorless liquid medium to provide a series of dilutions, or binary steps, of the odorant. For each successive binary step up the dilution scale, the odorant is present, for example, at one half the concentration of the preceding step. The highest concentration of the odorant usually provides the substance at an irritant level. The individual is presented with the series of dilutions in ascending order, and is asked to compare each dilution step to at least one control stimulus, such as odorless propylene glycol.

In the art, a “normosmic” individual is one who can detect the odor of a substance without irritant sensations when the odorant is presented with the range of its average normal threshold. A “hyposmic” or “microsmic” individual has reduced capacity of the olfactory nerve being able to detect an odorant substance by its odor at a concentration, or decismel level, above that of a normosmic individual yet below its irritant concentration level. An “anosmic” individual is one who has essentially no olfactory nerve capacity being unable to detect the odor of the odorant substance, but has trigeminal nerve function, being able to detect an odorant substance by means of irritant, tingling sensations when it is present at an irritant concentration. A patient who is able to detect pyridine vapor by means of irritant, tingling sensations caused by stimulation of the trigeminal nerve, but who cannot distinguish a pyridine odor at a lower concentration without such sensation, is considered to be anosmic having no olfactory nerve sensitivity.

Ranges of the average normal threshold for various odorant substances can be found in the art, for example, Amoore and O'Neill, “Proposal for Unifying Scale to Express Olfactory Thresholds and Odor Levels: The “Decismel Scale”,” in Proceedings of the 1988 Air Pollution control Association Annual Meeting, Paper No. 78.5 (21 pp.), Air and Waste Management Association, Pittsburgh, Pa. (1988); Amoore and Haotala, “Odor as an Aid to Chemical Safety: Odor Thresholds Compared with Threshold Limit Values and Volatiles for 214 Industrial Chemicals in Air and Water Dilution,” J. Appl. Toxicology 3(6):272-290 (1983).

A suprathreshold amount is a concentration of the odorant/odorant mixture that is greater than the average normal threshold concentration of the odorant or mixture. The normal threshold concentration can be determined by administering a series of the same concentrations of the odorant/odorant mixture to a control group of at least 25 individuals who do not have a chemosensory dysfunction, and calculating the mean threshold concentration detected by the group of 25 individuals. Another alternative is to refer to the known threshold concentration value for the odorant/odorant mixture that has been established previously and published by J. Amoore et al., J. Appl. Toxicology, 3:272 (1983).

Odor thresholds can be expressed on the decismel scale. The decismel scale is constructed by setting the mean threshold concentration of a chemosensory agent detected by the control group of 20 year olds at the “0” value. A decismel is calculated by dividing the concentration of the odorant detected by the patient by the normal threshold concentration (using the published value or empirically determining the value) and then taking the logarithm of the quotient. The logarithm of the quotient is then multiplied by 20 to obtain the decismel value. Decismel values can be positive or negative. A positive decismel value indicates the patient is less sensitive to the odorant, i.e. has a higher threshold detection concentration. A negative decismel value indicates that the patient is more sensitive to the compound, i.e. has a lower threshold detection concentration. An increase in the threshold concentration value over the mean threshold concentration value of 2 fold corresponds to 6 decismels (or ds). Determination of decismel units is known in the art, as addressed, for example, in U.S. Pat. Nos. 5,380,765 and 5,492,934 (Hirsch).

In another aspect, the invention provides odorant compositions that are considered to be hedonically positive by a subject individual and contain an effective amount of an odorant or mixture of odorants such that, when inhaled by the individual, the reaction time of the individual to a stimulus is substantially reduced (decreased) compared to their reaction time interval without having inhaled the odorant composition. Such an effect can be objectively assessed by measuring the reaction time of the individual to a stimulus both with and without the effect or inhalation of the odorant composition, for example, by measuring the period of time starting at the time of the exposure to the stimulus to the time of the reaction by the individual.

The concentration of the odorant or mixture of odorants is preferably at a suprathreshold concentration and preferably near but not an irritant concentration at a decismel level of about 25-55 decismel units, preferably greater than 25 decismel units, preferably at about 30-55 decismel units.

In embodiments of the invention, the hedonically pleasant/positive odorant composition contains an effective amount of a jasmine odorant as the dominant (primary) odor or essence such that, when inhaled, the subject individual's reaction time to a stimulus is reduced compared to the individual's reaction time to that stimulus without having inhaled the odorant composition. A preferred odorant composition for use in decreasing an individual's reaction time is a formulation in which the dominant odorant component is a jasmine odorant and eliminates odorants and other components that compete with the jasmine odorant accords or notes to provide a full effect on the individual inhaling the odorant composition. In some embodiments, the composition consists essentially of the jasmine odorant. In other embodiments, the jasmine odorant can be combined with secondary odorants that enhance but do not mask the jasmine odorant, or are hedonically neutral to the subject individual.

In embodiments of the invention, the odorant or odorant mixture is provided as a formulated composition of a single essential (primary or dominant) odorant or, in other embodiments as a blend (mixture) of two or more essential odorants, to cause the desired effect, and eliminates odorants and other components that compete with or mask the effective odorant(s). In some embodiments, the composition is composed of a blend of one or more essential odorants with one or more secondary odorants that do not compete with or mask the essential odorant(s), and/or are hedonically neutral to the subject individual, and in some embodiments enhance the essential odorant(s). The odorant composition can be formulated by combining the essential odorant(s) alone or as a blend with secondary odorant(s) with an odorless carrier such as mineral oil or water, and odorless additives such as preservatives and the like. The odorant composition can be formulated with a viscosity effective to allow for aerosolization or to provide a thick liquid material, e.g. a gel or cream.

In one embodiment, the composition can consist essentially of a suprathreshold and non-irritant concentration of one or more odorants such that, when inhaled by an individual, the composition is effective to modify (e.g., decrease) the reaction time of the individual to a stimulus by a statistically significant amount compared to the individual's reaction time to that stimulus without inhalation of the composition. For example, the composition can consist essentially of a jasmine odorant in a carrier with optional additives, such that, when inhaled by an individual, the composition is effective to decrease the individual's reaction time to a stimulus by a statistically significant amount compared to the individual's reaction time to the same stimulus without inhaling the composition. In another example, the composition can be composed of a mixture of odorants in a carrier with optional additives, including a suprathreshold and non-irritant concentration of a jasmine odorant in combination with a less than suprathreshold concentration of one or more odorants that complement and do not mask the jasmine odorant, such that, when inhaled by an individual, the composition is effective to decrease the individual's reaction time to a stimulus by a statistically significant amount compared to the individual's reaction time to the same stimulus without having inhaled the composition.

Odorants for use in the present methods, are commercially available as a liquid, essential oil, extract, or other form from a variety of sources, including, for example, Energy Essentials, AromaTech, Inc. (Somerville, N.J.), Florasynth, Inc. (Teterboro, N.J.), International Flavors and Fragrances, Inc. (IFF; New York, N.Y.), among others.

The odorant composition is preferably formulated as a liquid solution or a spray, but can also be provided in the form of a cream, lotion, or other consistency, and can be contained within a liquid pump device, aerosol or non-aerosol spray device, lidded container, a blister pack, or other suitable vessel such as those known and used in the art. The odorant composition can also be contained in a solid form within a capped vessel. It is preferred that the odorant composition is provided in a portable dispenser that is easily transportable and readily accessible.

In conducting the method of the invention, in some embodiments, the odorant composition is administered for inhalation by the subject individual prior to exposure to an external stimulus to increase the individual's reaction time to that stimulus. For example, to increase hand reflex reaction time, an odorant composition can be administered to an individual for inhalation and an object (e.g., pencil, stick, etc.) dropped between their grasp (fingers), which steps can be repeated. In some embodiments, the method can involve one or more sessions during which the individual inhales the odorant composition and is required to detect and make a quick or immediate decision or action in response to a command, question or other triggering event (e.g., a visual or auditory signal), and measuring and assessing the individual's reaction time. In conjunction with administering the odorant composition, the subject individual can be instructed as to the response to be made upon exposure to the external stimulus. The time period from initiation of the external stimulus to the time of the individual's response can be measured to provide the reaction time interval, which can be compared to a control and/or other time intervals measured for the same response to assess improvement in reaction time.

Administration of the odorant composition can be achieved by bringing an effective amount and concentration of the odorant composition into proximity of the individual for inhalation, for example, by spraying, by applying the odorant composition to a piece of clothing of the individual or directly to the face of the individual below the nostrils, or to a cloth or paper material such as a mask (e.g., a surgical mask, dust-type mask, earloop face mask, and the like) that is then secured over the nostrils of the subject individual, among other techniques. In another embodiment, the composition can be administered by means of a flexible laminate material (e.g., patch) sized to fit beneath the nose that incorporates the odorant composition and has a pressure-sensitive adhesive layer (covered by a release layer) that allows the material to adhere to skin and which is positioned under the nostrils of the individual, as described, for example in U.S. Pat. No. 6,769,428 (Cronk). In yet another embodiment, the odorant composition can be administered through the use of a portable delivery device operable to provide continuous delivery of a vaporous emission of the odorant composition through cannulla (tubes) inserted into the nostrils of the individual as described, for example, in U.S. Pat. No. 6,803,987 (Manne). Other delivery systems can be used for delivery of the odorant composition to the individual.

Odorants or odorant mixtures can be readily screened and assessed for effectiveness in modifying (e.g., reducing) an individual's reaction time to a stimulus according to the invention. For example, a composition containing an odorant or mixture of odorants can be administered to an individual for inhalation to evaluate its effect on modifying their reaction time to an external stimulus, which can be manifested, for example, by a decrease in the reaction time interval compared to the reaction time interval to the same stimulus without inhaling the odorant composition. In some embodiments, the individual can inhale an inactive odorant or neutral odorant as a control odorant composition. Elmes et al., Adequacy of Control Comparisons in Olfactory Experiments. Chem. Percept. 1:247-252 (2008). For example, an individual can be instructed to perform an action in response to an external stimulus and the interval of time between the stimulus and the performance of the action (without inhaling the test odorant composition) can be measured as a “control.” The subject individual can then inhale a test odorant composition, the stimulus can again be presented and the time interval between the stimulus and the action performed by the subject individual can be measured and compared to the control reaction time interval. Optionally, the individual can be questioned as to a positive or negative reaction to the pleasantness of the scent to assess the hedonics of the odorant composition.

Another embodiment of a method of screening a composition formulated with an odorant or a mixture of odorants for effectively enhancing an individual's reaction time can comprise the steps of:
a) having an individual perform an action in response to an external signal or stimulus without inhalation of the target odorant composition and measuring the time interval from the initiation of the stimulus to the subject individual's action as a “control” reaction time;
b) having the individual inhale a composition consisting essentially of the odorant or odorants to be tested, and then re-perform the same action in response to the same external stimulus from step a), and then measuring the interval of time from the initiation of the stimulus to the subject individual's action to provide a “test” reaction time;
c) comparing the control reaction time to the test reaction time to determine the statistical significance between the two reaction times; and
e) eliminating the odorant or odorant mixture as ineffective to enhance the individual's reaction time if not statistically significant (p<0.05).

The screening test as well as the method of the invention can include other steps such as having the inhaling individual identify the composition as hedonically positive, neutral or negative, and testing olfactory ability and/or olfactory capacity of the individual, among other olfactory tests known and used in the art.

According to the invention, a composition comprising the odorant or odorant mixture is administered (e.g., dispensed) as a vaporous emission to the nostrils of an individual for inhalation of a concentration of a primary odorant or odorant mixture effective to modify the individual's reaction time to an external stimulus, for example, using a jasmine odorant-based composition to decrease the individual's reaction time. Such an effect can be assessed and measured objectively by comparing the individual's reaction time to an external stimulus with and without the administration of the odorant composition.

The odorant composition can be packaged as part of an article of manufacture, or kit. In one embodiment, the article of manufacture can comprise a container of an odorant composition or, packaged together, a container of a first odorant and a container of a second odorant (etc.) for combining together to form the odorant composition. The odorant composition comprises an odorant or mixture of odorants in a suprathreshold and but non-irritant concentration, and preferably near a non-irritant concentration, effective to substantially modify an individual's reaction time to a stimulus when administered according to the method of the invention.

In a preferred embodiment, the composition consists essentially of a jasmine odorant. For example, the article of manufacture can comprise a container of an odorant composition consisting essentially of a jasmine odorant or of one or more odorants of which a jasmine odorant is the dominant (primary) odor or essence. The article of manufacture can further include a device for use in delivery of the composition to a subject individual, for example, a mask for placement over the nose of the individual, a device for applying the composition directly to the skin under the nostrils of the individual, a spray delivery device, among others.

The kit can further include one or more elements for testing the individual, that can be separately packaged, including a device for administering odorant(s) for testing olfactory ability of the individual (e.g., UPSIT), and/or a device for administering a series of odorants for testing olfactory threshold of the individual (e.g., pyridine dilution series).

The article of manufacture can further comprise written or other format of instructions (e.g., C.D., video, cassette tapes, etc.) for use of the odorant composition for modifying an individual's reaction time in a method according to the invention, including decreasing their reaction time to an external stimulus. The article of manufacture can further include one or more items for use in conducting the reaction test, for example a pencil/stick to be grasped, a ringer or bell, a timing device such as a stopwatch, and other devices. In another embodiment, the article of manufacture can comprise packaging material and an odorant composition according to the invention contained within the packaging material, wherein the packaging material comprises a label that indicates that the odorant composition can be used for modifying (e.g., decreasing) reaction time to an external stimulus. The article of manufacture can also include an odorant composition and instructions for testing olfactory threshold according methods known in the art. The parts of the article of manufacturing can be contained or separately packaged within a packaging material, such as a box, bag, pouch, and the like.

The invention will be further described by reference to the following detailed example. This example is not meant to limit the scope of the invention that has been set forth in the foregoing description. Variation within the concepts of the invention is apparent to those skilled in the art. The disclosures of the cited references throughout the application are incorporated by reference herein.

### EXAMPLE

Methods. Twenty-one subjects (15 males and 6 females) ranging from 18 to 61 years old, chosen using a convenience paradigm, participated in an Institutional Review Board (IRB) approved study to determine the effect of test odorants on reaction time. The Quick Smell Identification Test (Q-SIT), a three (3) question scratch-and-sniff smell identification test (Sensonics, Inc., Haddon Heights, N.J.) was administered to each subject as per manufacturer's direction to screen for gross olfactory deficits. Doty, R. L., Office Procedures for Quantitative Assessment of Olfactory Function, Am J Rhinol, 21, 4: 460-473 (2007).

Two test odors, a jasmine odor CE-44550 (Custom Essence) and a charcoal mesquite meat odor 2188-HS (International Flavors & Fragrance, Inc., New York, N.Y.) were tested due to their opposite hedonic characteristics, as determined by an internal panel at The Smell & Taste Treatment and Research Foundation (Chicago, Ill.). The test odors were impregnated upon 3M® Aseptex® molded surgical masks. The subjects were asked to identify each odor as hedonically neutral, pleasant or unpleasant. A 1-minute interval elapsed after placement of a mask and before the start of the testing to eliminate the novelty effect. Kane et al., Conceptual and Perceptual Novelty Effects in Human Medial Temporal Cortex, Hippocampus 15, 3, 326-332 (2004). Subjects underwent a no-odor, wash out interval before a new mask was introduced.

The reaction times of the subjects were measured while wearing the masks impregnated with the test odorants and a non-odorized mask as a control. Each subject served as his or her own control while wearing an unodorized mask. The test was conducted using an instrument and method described in Eckner et al., The Effect of Concussion on Clinically Measured Reaction Time in NCAA Division I Collegiate Athletes: A Pilot Study, Neurology 74, Mar. 2, 2010, Suppl. 2, #S17.002; and Eckner et al., Can a Clinical Measure of Reaction Time Predict a Functional Sport-Protective Response? Implications for Sport Concussion Management. Neurology 74, Mar. 2, 2010, Suppl. 2, #P02.029. The order of presentation of odors was randomized to eliminate any interaction due to learning. Subjects attended to a Queen Square Reflex Hammer-like device, which at random intervals would be dropped and the distance through which the instrument had fallen before it could be grasped was measured. After two practice trials, eight trials of reaction time testing were conducted and measurements of distance dropped before grasping were recorded and averaged. Reaction time was calculated using the formula:

$$Average reaction time ( milliseconds ) = 1000 * \sqrt{\frac{2 * distance}{980}}$$

The results were independently analyzed using ANOVA. The statistical significance was defined at p<0.05. Compared to the control, each odor was assessed and independently analyzed based on order of presentation, hedonics and gender, using ANOVA.

A total of 21 subjects were exposed to three different odors and the calculated reaction times are shown in Table 1 below. (Odor #0 is Blank (control); Odor #1 is Jasmine CE-44550; Odor #2 is Charcoal Mesquite Meat Combination fragrance 2188-HS.)

TABLE 1

| | | | | | | Hedonics¹ | | | Average | | |
|---|---|---|---|---|---|---|---|---|---|---|---|
| No. | Sex | Age | Smoker | QSIT³ | Order⁴ | Hed#0 | Hed#1 | Hed#2 | Avg#0 | Avg#1 | Avg#2 |
| 1 | F | 39 | N | 3 | 132 | N | P | U | 28.06 | 22.5 | 31.5 |
| 2 | M | 45 | Y | 2 | 312 | N | N | N | 16.37 | 15.37 | 27.37 |
| 3 | M | 28 | N | 3 | 231 | N | N | N | 24.37 | 18.06 | 28.18 |
| 4 | F | 46 | N | 3 | 132 | N | N | P | 21.87 | 25.62 | 28.37 |
| 5 | F | 24 | N | 3 | 213 | N | P | N | 27.5 | 28.62 | 34.82 |
| 6 | M | 39 | N | 3 | 312 | N | P | U | 22.62 | 18.62 | 30.87 |
| 7 | F | 22 | Y | 2 | 123 | N | N | N | 30.62 | 19.62 | 18.5 |
| 8 | F | 28 | N | 3 | 123 | N | P | U | 27.25 | 21.75 | 25.93 |
| 9 | M | 33 | N | 3 | 123 | N | P | N | 24.12 | 17.75 | 23.93 |
| 10 | M | 27 | N | 3 | 321 | N | N | U | 20.75 | 19.75 | 26.5 |
| 11 | M | 35 | N | 1 | 312 | N | P | P | 22.12 | 17.62 | 23 |
| 12 | M | 61 | Y | 2 | 213 | N | P | U | 32 | 31.12 | 22.25 |
| 13 | M | 18 | N | 2 | 123 | N | P | N | 22.31 | 17.06 | 16 |
| 14 | M | 50 | N | 3 | 213 | N | P | P | 28.25 | 29.6 | 25.37 |
| 15 | M | 56 | N | 2 | 231 | N | N | U | 20.12 | 23.12 | 31.37 |
| 16 | M | 52 | N | 3 | 213 | N | P | U | 16.75 | 13.37 | 16.75 |
| 17 | M | 56 | N | 3 | 123 | N | P | U | 28.31 | 22.06 | 27.68 |
| 18 | M | 24 | N | 3 | 312 | N | P | N | 23.5 | 21.24 | 23.82 |
| 19 | F | 56 | N | 3 | 312 | N | P | U | 27.37 | 23.62 | 28.3 |
| 20 | M | 50 | N | 3 | 231 | N | P | U | 25.5 | 25.12 | 26.62 |
| 21 | M | 25 | N | 3 | 321 | N | P | U | 16.8 | 15.93 | 21.5 |

¹Hedonics: N = Neutral, P = Pleasant, U = Unpleasant

TABLE 2
Reaction time by odor (n = 21)

| Odor | Mean | SD | p-value¹ |
|---|---|---|---|
| Blank (control) | 220.9 | 20.9 | |
| Jasmine | 207.3 | 23.2 | 0.0014 |
| Charcoal Mesquite Meat | 227.7 | 22.3 | 0.2752 |
| Combo | | | |

¹The p-value for contrast to blank (control) within-subjects effects in
repeated measures ANOVA.

| Odor | Mean | SD | p-value¹ |
|---|---|---|---|
| Neutral | 220.9 | 20.9 | |
| Jasmine & Charcoal | 217.5 | 19.4 | .4388 |
| Mesquite Meat | | | |
| Combined | | | |

¹The p-value for contrast to Neutral, within-subjects effects in repeated
measures ANOVA.

TABLE 3
By Order of Presentation (n = 21)

| Order | Mean | SD | p-value¹ |
|---|---|---|---|
| First | 226.6 | 21.3 | |
| Second | 218.3 | 25.1 | .1832 |
| Third | 211.1 | 22.2 | .0123 |

¹The p-value for contrast to First, within-subjects effects in repeated measures ANOVA.

TABLE 4
By hedonics (n = 16). Subjects reporting both neutral and pleasant odors.

| Average for all odors | | | |
|---|---|---|---|
| that test subject found to be: | Mean | SD | p-value ¹ |
| Blank (control) | 223.9 | 21.4 | |
| Pleasant | 211.4 | 24.3 | .0015 |

¹ The p-value for contrast to blank (control), within-subjects effects in repeated measures ANOVA.

TABLE 5
Subjects reporting both neutral and unpleasant odors (n = 11)

| Average for all odors | | | |
|---|---|---|---|
| subject found to be: | Mean | SD | p-value¹ |
| Blank (control) | 221.2 | 23.4 | |
| Unpleasant | 230.8 | 21.1 | .2138 |

¹The p-value for contrast to blank (control), within-subjects effects in repeated measures ANOVA.

TABLE 6
Average measures of reaction time by gender

| | |
|---|---|
| Males (n = 15) | Females (n = 6) |

| Odor | Mean | SD | Mean | SD |
|---|---|---|---|---|
| Blank (control) | 215.3 | 21.2 | 234.9 | 12.7 |
| Jasmine | 202.6 | 24.7 | 219.2 | 14.5 |
| Charcoal | 223.8 | 20.9 | 237.6 | 24.7 |
| Average | 213.9 | | 230.6 | |

¹The p-value for contrast to blank (control), within-subjects effects in repeated measures ANOVA.

Results of repeated measures ANOVA:

Main effect of gender (between-subjects): F=4.48, df=1, p=0.0478

Interaction between effect of odor and gender: F=0.13, df=2, p=0.8554

Results/Discussion. The results showed that overall, a hedonically pleasant aroma (jasmine) decreased reaction time while aromas perceived as hedonically unpleasant (charcoal mesquite meat) increased reaction time. In the Example, jasmine reduced reaction time as compared to the blank (neutral) and the charcoal mesquite meat (barbeque) odor.

Subjects had faster reaction times when exposed to jasmine (reaction time 207.3 ms) compared to the blank (neutral) neutral mask (220.9 ms) (p=0.0014), and when exposed to odors they found to be pleasant (211.4 ms) compared to the blank (neutral) mask (223.9 ms) (p=0.0015). Reaction times of subjects exposed to the charcoal mesquite meat odor (227.7 ms) and odors found to be unpleasant (230.8 ms) had slightly increased reaction times, but not significantly (p=0.2752 and p=0.2138, respectively).

An effect of order presentation was demonstrated with each series of presentations from 226.6 ms to 218.3 ms to 211.1 ms. Average reaction times decreased slightly from first to second series (p=0.1832) and decreased significantly from the 1st to the 3rd series (p=0.0123). Men demonstrated a faster reaction time than women (213.9 ms versus 230.6 ms) (p=0.0478; p=0.8554) but the pattern of responses to the different odors was the same for both genders (p=0.8554 for interaction between gender and odor).

The results demonstrated that an individual's reaction time can be decreased by administering an odorant that is hedonically pleasant to the individual. The results further demonstrated that inhalation of a jasmine odorant had a significant effect on decreasing an individual's reaction time.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents. The disclosures of the cited patents, applications, and other references throughout the application are incorporated by reference herein.

What is claimed is:

1. A method of modifying an individual's reaction time, comprising:
administering to the individual for inhalation an effective amount of a composition comprising an odorant such that the individual's reaction time is modified by a statistically significant amount compared to the individual's reaction time without inhalation of the composition.

2. The method of claim 1, wherein the composition comprises an effective amount of a jasmine odorant to decrease the individual's reaction time, in an odorless carrier.

3. The method of claim 2, wherein the composition consists essentially of the jasmine odorant in a carrier, and optional additives.

4. The method of claim 1, wherein the composition comprises a mixture of odorants in a carrier, said mixture of odorants comprising a suprathreshold and non-irritant concentration of a jasmine odorant in combination with a less than suprathreshold concentration of one or more odorants that complement and do not mask the jasmine odorant, such that, when inhaled by an individual, the composition is effective to decrease the individual's reaction time by said statistically significant amount.

5. The method of claim 1, wherein the composition comprises a concentration of the odorant greater than an average normal threshold concentration of the odorant, the concentration being about 25-55 decismel units.

6. The method of claim 1, wherein the composition comprises a suprathreshold but non-irritant concentration of the odorant.

7. The method of claim 1, further comprising assessing said reaction times to determine effectiveness of administering the composition to modify the individual's reaction time.

8. An article of manufacture, comprising, packaged together:
a container of a composition comprising an odorant; and
instructions for administration of the composition to an individual by inhalation for modifying the individual's reaction time.

9. The article of claim 8, further comprising at least one of the following elements:
a device for administering a plurality of odorants for testing olfactory ability of the individual; and
a device for administering a series of odorants for testing olfactory threshold of the individual.

10. The article of claim 8, further comprising, packaged with said container and the instructions, at least one of:
a set of odorants for testing an individual's olfactory ability; and
a set of serial dilutions of an odorant for testing olfactory threshold.

11. A method of screening a test odorant composition consisting essentially of one or more test odorants, for effectively altering an individual's reaction time to an external stimulus, comprising:
a) instructing an individual to perform an action upon detecting an external stimulus;
b) initiating the external stimulus to the individual without the presence of the odorant composition;
c) measuring the interval of time between said initiation of the external stimulus and the performance of the action as a “control” reaction time;
d) having the individual inhale the test odorant composition;
e) initiating said external stimulus to the individual;
f) measuring the interval of time between said initiation of the external stimulus in step e) and the performance of said action to provide a “test” reaction time;
g) comparing the control reaction time to the test reaction time to determine the statistical significance between the two reaction times; and
h) eliminating the odorant composition as being ineffective to modify the individual's reaction time if not statistically significant.

12. The method of claim 11, wherein inhaling the odorant composition reduces the reaction time interval compared to the control reaction time interval.

13. The method of claim 11, further comprising, prior to step b), having the individual inhale an inactive odorant as a control odorant composition.

14. A method of screening an odorant composition consisting essentially of the odorant or odorants to be tested for enhancing an individual's reaction time, comprising the steps of:
a) having the individual perform an action in response to an external stimulus without inhalation of the odorant composition and measuring the interval of time between initiation of the external stimulus and performance of the action as a “control” reaction time;
b) having the individual re-perform said action in response to said external stimulus while inhaling the odorant composition and measuring the interval of time between initiation of the external stimulus and performance of the action as a “test” reaction time;
c) comparing the control reaction time to the test reaction time to determine the statistical significance of the test reaction time;
d) assessing the composition as effective to enhance the individual reaction time based on said statistical significance of the test reaction time; and
e) eliminating the odorant composition as ineffective to enhance the individual's reaction time if not statistically significant (p<0.05).
